(19) United States
(12) Patent Application Publication
(10) Pub. No.: US 2021/0093490 A1
(43) Pub. Date: Apr. 1, 2021

(54) COMPOSITE WEBS

(71) Applicant: ATTENDS HEALTHCARE PRODUCTS, INC., Raleigh, NC (US)

(72) Inventor: Charles F. SCHROER, Raleigh, NC (US)

(21) Appl. No.: 16/955,636

(22) PCT Filed: Dec. 17, 2018
(86) PCT No.: PCT/US2018/066046

§ 371 Date: Jun. 18, 2020

Related U.S. Application Data
(60) Provisional application No. 62/609,018, filed on Dec. 21, 2017.

(51) Int. Cl. A61F 13/538; A61F 13/496; A61F 13/511; D21H 27/00; D21H 21/22; D21H 17/20; D03D 15/46; D03D 1/00; D03D 15/56

(52) U.S. Cl. CPC A61F 13/538; A61F 13/496; A61F 13/511; D21H 27/002; A61F 2013/530664; D21H 17/20; D03D 15/46; D03D 1/00; D03D 15/56; D21H 21/22

(57) ABSTRACT

Composite webs comprising a plurality of first strips of tissue or nonwoven material and a plurality of second strips oriented at a non-parallel angle relative to the first strips.

### CROSS-REFERENCE TO RELATED APPLICATIONS

This application claims priority to and the benefit of U.S. Provisional Application No. 62/609,018, filed Dec. 21, 2017, the contents of which is incorporated by reference into the present application.

### FIELD OF INVENTION

The present invention relates generally to sheet and laminate materials such as those used for absorbent products like adult incontinence briefs, protective underwear, feminine hygiene pads, and infant diapers, training pants, and the like; and more particularly, but not by way of limitation, to composite webs that can be used in such products.

### BACKGROUND

Examples of sheet and laminate materials used for absorbent products include laminates and nonwoven materials, such as nonwoven absorbent substrates. The process by which such substrates are made typically defines the properties of the substrate, for example flexibility, fluid absorption characteristics, and fluid movement characteristics. Generally speaking, absorbent products typically include an absorbent core sandwiched between a topsheet and a backsheet, and may also include one or more multi-layered laminates with multiple layers, one on another, with adhesive in between layers.

Absorbent products that may include laminates and nonwoven materials include disposable absorbent articles that are wearable by a user, examples of which include baby diapers, training pants, and adult incontinence briefs and underwear, all of which may be made in disposable forms. “Disposable” refers to articles that are designed to be discarded after a limited use rather than being laundered or otherwise restored for reuse. Disposable absorbent products have met with widespread acceptance in the marketplace for a variety of applications, including infant and adult incontinence care, in view of the manner in which such products can provide effective and convenient liquid absorption and retention while maintaining the comfort of the wearer. Such disposable absorbent articles often include a topsheet that is configured to be closest to the wearer during use, a liquid-impermeable backsheet or outer cover, and an absorbent core between the topsheet and the backsheet. In some instances, such disposable absorbent articles also include an acquisition-distribution layer (ADL) disposed between the topsheet and the absorbent core. Elasticated standing leg cuffs and leg gathers are also often used in such articles to provide improved fit and reduced leakage around a wearer's legs, relative to articles without such cuffs or gathers.

U.S. Pat. No. 4,670,011 discloses certain prior art examples of diapers, and U.S. Pat. Nos. 6,976,978 and 4,940,464 disclose certain prior art examples of disposable incontinence garments or training pants.

One example of such a disposable absorbent article is shown in FIGS. 1A-1B, which depict a lower plan view and a perspective view, respectively, of adult protective underwear 10. Underwear 10 includes a chassis 14 having a front waist portion 18, an opposing rear waist portion 22, and a crotch portion 26 extending longitudinally between front and rear waist portions 18, 22. Chassis 14 further includes a backsheet 30 defining an outer surface and configured to face away from a wearer during use of the diaper, and topsheet 34 defining an opposing body facing surface and configured to face a wearer during use of the diaper.

As shown in FIGS. 1A-1B, underwear 10 further includes a pair of front elastic side panels 38 and a pair of rear elastic side panels 42 configured to couple rear waist portion 22 to front waist portion 18 in a well-known configuration in which a left side 46 of the chassis defines a first leg opening 50 for a wearer's left leg, and in which a right side 54 of the chassis defines a second leg opening 58 for the wearer's right leg. In the depicted configuration, each of side panels 38, 42 includes a connection portion 62 configured to be coupled to a connection portion 62 of another of side panels 38, 42. Specifically, connection portion 62 of the left one of front side panels 38 is configure to be coupled to connection portion 62 of the left one of rear side panels 42, and connection portion 62 of the right one of front side panels 38 is configure to be coupled to connection portion 62 of the right one of rear side panels 42, such that the waist portions 18, 22 and side panels, 38, 42 cooperate to define a waist opening 66 as shown in FIG. 1B. Connection portions 62 of the respective side panels can be permanently coupled together to define a tear-able side seam 70, such as, for example, via adhesive, ultrasonic, or thermal bonds. Such tear-able side seams generally cannot be refastened, and thereby render an article unusable once opened. Alternatively, connection portions 62 of the respective side panels can be removably coupled to define a refastenable or adjustable side seam, such as, for example, via hook-and-loop fasteners. Hook and loop fasteners are mechanical fasteners that include hooks, such as in a hook fastener portion, that are configured to engage loops in a loop fastener portion or in fibers of a sheet of fabric; for example, a nonwoven or woven fabric with fibers that define open or loop-like regions into which the hooks can extend and engage. Examples of such hook and loop fasteners may be referred to as VELCRO.

As is known in the art, underwear 10 can include one or more elastic elements coupled to the chassis such that the one or more elastic elements resist expansion of a circumference of the first leg opening and resist expansion of a circumference of the second leg opening. For example, as shown in FIG. 1A, the depicted embodiment of the chassis (14) includes a first elastic region 74 along right side 46, and a second elastic region 78 along left side 54. In some configurations, elastic regions 74, 78 can each be defined by one or more elastic strands, which may be referred to in the art as “leg elastics,” coupled to the chassis, for example laminated between the topsheet or an additional leg cuff layer and the backsheet. In other configurations, elastic regions 74, 78 can each be defined by an elastic film coupled to the chassis, for example laminated between the topsheet and the backsheet. In configurations in which elastic regions 74, 78 are defined by elastic film, the regions can be defined by separate pieces of elastic film or by separate regions of a single piece of elastic film. As shown in FIG. 1A, elastic regions 74, 78 may be parallel to and/or extend along a majority of a length of each of sides 46 and 54, provided that the elastic regions are configured to provide a biasing force that resists expansion of the leg openings when the chassis is in its closed configuration and tends to contract the leg opening around a wearer's leg, as shown in FIG. 1B. Contraction of the leg opening to conform to the wearer's leg is desired for good containment of urine and feces in an absorbent product.

Another example of such a disposable absorbent article is shown in FIGS. 2A and 2B, which depict lower plan views of an adult incontinence brief 100. Brief 100 includes a chassis 104 having a front waist portion 108, an opposing rear waist portion 112, and a crotch portion 116 extending longitudinally between front and rear waist portions 108, 112. Chassis 104 further includes an outer surface 128 configured to face away from a wearer during use of the diaper, and an opposing body facing surface 132 configured to face a wearer during use of the diaper. In the view of FIG. 2A, a dashed leader extends from the body facing surface to reference numeral 132 because body facing surface 132 is opposite outer surface 128 and therefore not visible in the view of FIG. 2A.

As shown in FIG. 2A, brief 100 further includes a pair of closure members 136 configured to couple rear waist portion 112 to front waist portion 108 in a well-known configuration in which a left side 140 of the chassis defines a first leg opening for a wearer's left leg, and in which a right side 144 of the chassis defines a second leg opening for the wearer's right leg, similar in some respects to what is shown in FIG. 1B for training pant 10. In the depicted configuration, the closure members include a pair of back ears or back ear panels 148 each having a first end 152 bonded to rear waist portion 112 of chassis 104, and a second end 156 shown extending away from rear waist portion 112. “Bonded” refers to the joining, adhering, connecting, attaching, or the like, of two elements via adhesive(s), ultrasonic bond(s), and/or thermal bond(s). Two elements will be considered to be bonded together when they are bonded directly to one another or indirectly to one another, such as when each is directly bonded to intermediate elements.

Each closure member 136 further includes a fastener tab 160 with a first end 164 bonded to back ear 148, a second end 168 shown extending laterally outward from back ear 148, and a fastener portion 172 coupled to the fastener tab. Back ears 148 are each formed of a stretchable elastic material, such as a nonwoven laminate, that permits adjustments in the width and tension of back ears 148 to vary the form and fit of brief 100 when worn by a user.

Fastener tabs 160 are formed of an inelastic nonwoven material and carry fastener portions 172. Fastener portions 172 include strips of hook material configured to interact with a corresponding loop material in the well-known hook-and-loop fastener arrangement. Connection of closure members 136 to front waist portion 108 is facilitated by a landing zone 176 configured to be engaged by fastener portions 172. In this embodiment, landing zone 176 is defined by an anchoring member that includes a strip of loop material bonded to front waist portion 108 of chassis 104, for example, to the backsheet, and configured to be engaged by the hook material of fastener portions 172.

As shown in FIG. 2A, brief 100 also includes a pair of front ears 180 extending from opposite sides 140, 144 of chassis 104 with each of front ears 180 each having a first end 184 bonded to front waist portion 108 of chassis 104, and a second end 188 shown extending away from a respective side of front waist portion 108. Front ears 180 are each formed of a relatively soft nonwoven material and are each configured to be overlapped by the corresponding fastener tab 160 and/or back ear 148 to prevent the edges of fastener tab 160 from pinching, rubbing, or otherwise irritating a user's skin in use when fastening portions 172 are engaged with landing zone 176 to couple rear waist portion 112 to front waist portion 108. In some embodiments, front ears 180 include loop fastener portions or a fabric that is configured to be engaged by hook fastener portions such that fastener portions 172a can engage front ears 180.

Outer surface 128 is defined by a liquid-impermeable backsheet or cover 192 that defines outer surface 128, and a liquid-permeable topsheet 196 that defines body facing surface 132 and is configured to be closest to the wearer during use. “Liquid impermeable,” when used in describing a layer or multi-layer laminate, means that a liquid, such as urine, will not pass through the layer or laminate, under ordinary use conditions, in a direction generally perpendicular to the plane of the layer or laminate at the point of liquid contact. “Lamination” is the technique of manufacturing a material in multiple layers, so that the composite material has benefits of all the combined layers, such as, for example, improved mechanical strength or durability, improved stability, lower permeability to water, and/or other properties. A laminate includes two or more layers of material(s) that are permanently assembled by heat, pressure, ultrasonic welding, or adhesives.

As shown in FIG. 2B, the depicted embodiment include an absorbent core 200 disposed between topsheet 196 and backsheet 192. An “absorbent core” is a structure typically disposed between a topsheet and backsheet of an absorbent article and containing materials like SAP and/or cellulosic fibers that are configured to absorb liquid in the absorbent article.

As shown in FIG. 2B, brief 100 also includes an acquisition-distribution layer (ADL) 204 disposed between the topsheet and the absorbent core. “Layer” when used in the singular can be a single element or a plurality of elements. For example, a plurality of sheets may together define a single layer, such as, for example, a layer with a particular function to which the sheets of the layer contribute.

As is known in the art, brief 100 can include one or more elastic elements coupled to the chassis such that the one or more elastic elements resist expansion of a circumference of the first leg opening and resist expansion of a circumference of the second leg opening. For example, as shown in FIG. 2B, the depicted configuration of the chassis (104) includes a first elastic region 208 along first side 140, and a second elastic region 208 along second side 140. In some configurations, elastic regions 208 can each be defined by one or more elastic strands, which may be referred to in the art as “leg elastics,” coupled to the chassis, for example laminated between the topsheet (or an additional leg cuff layer) and the backsheet. In other configurations, elastic regions 208 can each be defined by an elastic film coupled to the chassis, for example laminated between the topsheet (or an additional leg cuff layer) and the backsheet. In configurations in which elastic regions 208 are defined by elastic film, the regions can be defined by separate pieces of elastic film or by separate regions of a single piece of elastic film. As shown in FIG. 2B, elastic regions 208 may be parallel to and/or extend along a majority of a length of each of sides 140 and 144, provided that the elastic regions are configured to provide a biasing force that resists expansion of the leg openings when the chassis is in its closed configuration.

As shown in FIG. 2A, chassis 104 has an overall relaxed length 212.

Brief 100 of FIGS. 2A and 2B is typically packaged and sold in a folded, and unfastened configuration in which chassis 104 is folded in half such that rear waist portion 108 overlaps front waist portion 104, but fastener portions 172 do not engage landing zone 176. While brief 100 is described as an adult incontinence brief, brief 100 can also comprise a baby diaper or training pant.

The materials that comprise underwear 10 of FIGS. 1A and 1B and brief 100 of FIGS. 2A and 2B, such as nonwoven absorbent substrates, have properties defined by the process by which such materials are made. For example, some nonwoven absorbent substrates are stretchable only in one direction. Some substrates have high liquid-absorption capacities, but cannot effectively transport absorbed liquid beyond the insult point of the liquid. Yet other substrates may effectively transport liquid, but are stiff and have lower absorption capacities. Further processing is required to enhance the properties of the substrate, for example to achieve more desirable fluid management characteristics, including chemical treatment, embossing, layering, cutting, coating, and/or impregnating the substrate. However, multi-layered laminates, having one substrate adhered to another may be relatively stiff and may have limited flexibility for comfort, as well as limited fluid management capabilities due to layer-to-layer construction.

### SUMMARY

Accordingly, there is a need in the art for composite webs that have more desirable properties than webs made solely of their constituent substrates. Providing such composite webs addresses the above-noted limited properties of nonwoven absorbent substrates, laminates, and other materials used for absorbent products. This disclosure includes embodiments of composite webs, methods of making composite webs, methods of making disposable absorbent articles including such composite webs, and such disposable absorbent articles, for example, adult incontinence briefs and protective underwear with leg openings that are adjustable for improved fit around a wearer's legs. The present substrates can be made with properties that are better suited for certain applications in absorbent articles than are existing substrates. For example, the present substrates combine desirable properties of different webs, for example tissue, laminates, and/or nonwovens, into a single composite web of material.

Some embodiments of the present composite webs comprise: a plurality of first strips of tissue, paper, or nonwoven web extending in a first direction and a plurality of second strips extending in a second direction disposed at a non-parallel angle relative to the first direction. In some embodiments, the second direction is perpendicular to the first direction. In some embodiments of the present composite webs, the plurality of first strips and the plurality of second strips are woven together. In some embodiments, the first strips are bonded to the second strips.

In some embodiments of the present composite webs, the first strips comprise a laminate comprising tissue and superabsorbent polymer particles. In some embodiments, the second strips comprise a laminate comprising tissue and superabsorbent polymer particles. In other embodiments, the second strips comprise a through-air bonded polymer nonwoven web. In some embodiments, each of the second strips comprises a plurality of channels extending along the second direction. In some embodiments, the first strips are configured to stretch more in the first direction than a direction perpendicular to the first direction. In some embodiments, the second strips are configured to stretch more in the second direction than in a direction perpendicular to the second direction. In some embodiments, the first strips are at least 5% thicker than the second strips.

Some embodiments of the present composite webs further comprise a plurality of third strips extending in a third direction disposed at a non-parallel angle relative to the first direction. In some embodiments, the first strips and the third strips comprise a laminate comprising tissue and superabsorbent polymer particles. In some embodiments, the third direction is the same as the second direction. In some embodiments, the first, second, and third strips are woven together such that the composite web comprises a weave having a warp comprising the first strips and a weft comprising the second and third strips. In some embodiments, the weft of the weave alternates between second strips and third strips such that each of the second strips is adjacent to at least one of the third strips.

Some embodiments of the present absorbent articles comprise a chassis having opposing front and rear waist portions, a crotch portion extending longitudinally between the front and rear waist portions, where a first end of the front waist portion is configured to be coupled to a first end of the rear waist portion and a second end of the front waist portion is configured to be coupled to a second end of the rear waist portion to define a closed configuration in which the front and rear waist portions cooperate to encircle and define a waist opening, a left side of the chassis defines a first leg opening, and a right side of the chassis defines a second leg opening. In some embodiments of the present absorbent articles, the chassis comprises at least one of the present composite webs.

In some embodiments of the present absorbent articles, a topsheet is disposed on at least one of the present composite webs. In some embodiments, a topsheet is disposed on at least one of the present composite webs such that first strips of the composite web swell to a first thickness when the first strips absorb moisture and the first strips and the second strips are woven together such that the topsheet contacts the first strips and not the second strips when the first strips have the first thickness. Some embodiments of the present absorbent articles further comprise a liquid impermeable backsheet disposed on at least one composite web. In some embodiments, the chassis comprises a composite web having first and second strips comprising paper.

In some embodiments of the present methods of making a composite web, the method comprises orienting a plurality of first strips in a first direction and coupling a plurality of second strips to the first strips in a second direction disposed at a nonparallel angle relative to the first direction. In some embodiments, the second strips are woven between the first strips such that the first and second strips are woven together. In some embodiments, the first strips comprise warp strips and the second strips comprise one or more continuous weft strips, and the second strips are woven between the first strips in a series of passes. In some embodiments, the first strips are bonded to the second strips. In some embodiments of the present methods, the first strips comprise tissue or nonwoven web. In some embodiments, the first strips are produced by cutting a first sheet comprising tissue or a nonwoven web. In some embodiments, the second strips are produced by cutting a second sheet comprising tissue or a nonwoven web.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified—and includes what is specified; e.g., substantially 90 degrees includes 90 degrees and substantially parallel includes parallel—as understood by a person of ordinary skill in the art. In any disclosed embodiment, the term “substantially” may be substituted with “within [a percentage] of” what is specified, where the percentage includes 0.1, 1, 5, and 10 percent.

The terms “comprise” and any form thereof such as “comprises” and “comprising,” “have” and any form thereof such as “has” and “having,” and “include” and any form thereof such as “includes” and “including” are open-ended linking verbs. As a result, an apparatus that “comprises,” “has,” or “includes” one or more elements possesses those one or more elements, but is not limited to possessing only those elements. Likewise, a method that “comprises,” “has,” or “includes” one or more steps possesses those one or more steps, but is not limited to possessing only those one or more steps.

Any embodiment of any of the apparatuses, systems, and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Further, a device or system that is configured in a certain way is configured in at least that way, but it can also be configured in other ways than those specifically described.

The feature or features of one embodiment may be applied to other embodiments, even though not described or illustrated, unless expressly prohibited by this disclosure or the nature of the embodiments.

Some details associated with the embodiments described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. Views in the figures are drawn to scale, unless otherwise noted, meaning the sizes of the depicted elements are accurate relative to each other for at least the embodiment in the view.

FIG. 1A depicts a bottom plan view of a prior art disposable absorbent article, specifically adult protective underwear, in an open configuration.

FIG. 1B depicts a perspective view of the protective underwear of FIG. 1A in a closed configuration.

FIG. 2A depicts a bottom plan view of a prior art disposable absorbent article, specifically an adult incontinence brief, in an open configuration.

FIG. 2B depicts a bottom plan view of the brief of FIG. 2A, in an open configuration, showing certain internal components of the brief.

FIG. 3A depicts a plan view of a first embodiment of the present composite webs, specifically a woven composite web before the composite web is incorporated into an absorbent article.

FIG. 3B depicts a high loft laminate used in the composite web of FIG. 3A.

FIG. 3C depicts an embossed laminate used in the composite web of FIG. 3A.

FIG. 4 depicts a plan view of a second embodiment of the present composite webs, specifically a woven composite web before the composite web is incorporated into an absorbent article.

FIG. 5A depicts a plan view of a third embodiment of the present composite webs, specifically a woven composite web before the composite web is incorporated into an absorbent article.

FIG. 5B depicts the woven composite web of FIG. 5A.

FIG. 6A depicts a first technique of making the woven composite web of FIG. 3A, specifically a plan view of a plurality of strips being woven together.

FIG. 6B depicts the woven composite web of FIG. 3A, specifically after the plurality of strips of FIG. 6A are woven together.

FIG. 6C depicts a close-up view of the woven composite web of FIG. 3A, using the embossed laminate of FIG. 3C and the high loft laminate of FIG. 3B.

FIG. 6D depicts a second technique of making a woven composite web.

FIG. 7 depicts a perspective view of an embodiment of the present absorbent articles, specifically an adult incontinence brief in the closed position that incorporates a composite web.

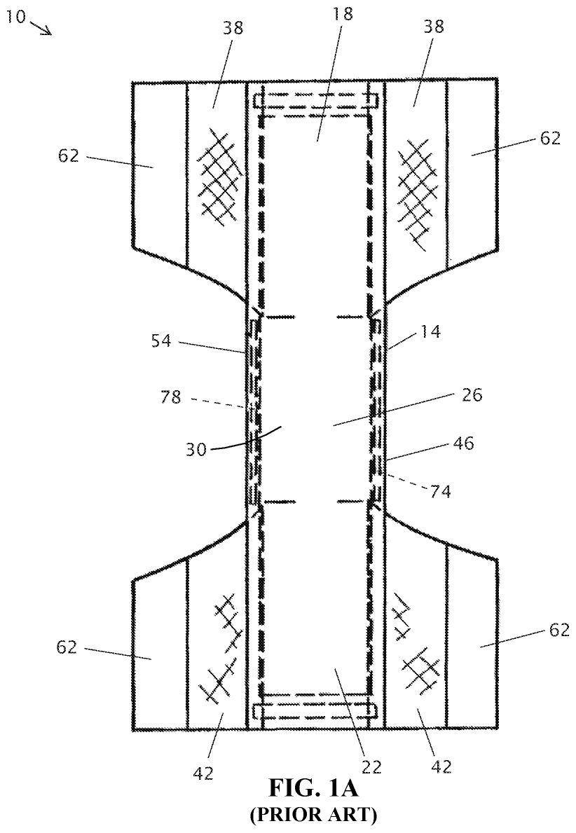

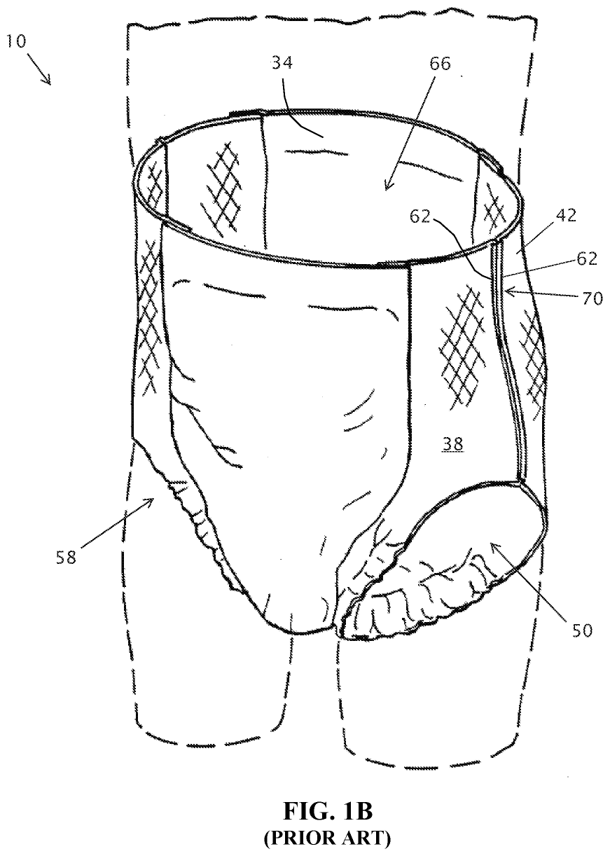

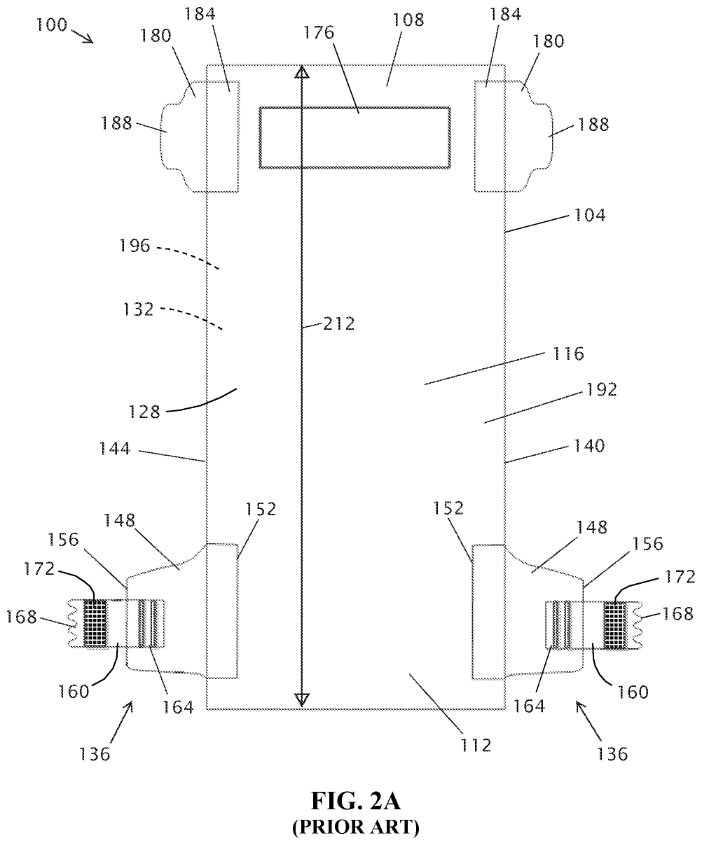

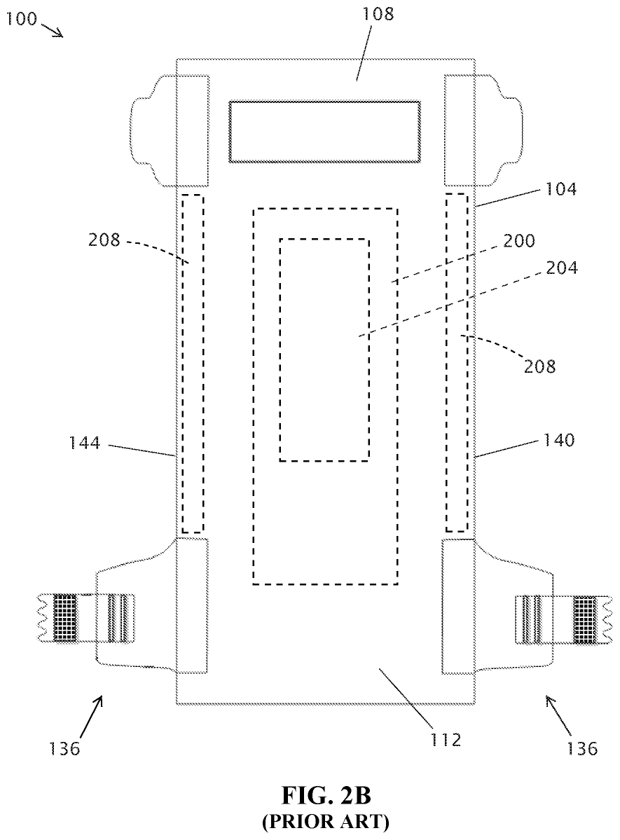

### DETAILED DESCRIPTION OF ILLUSTRATIVE EMBODIMENTS

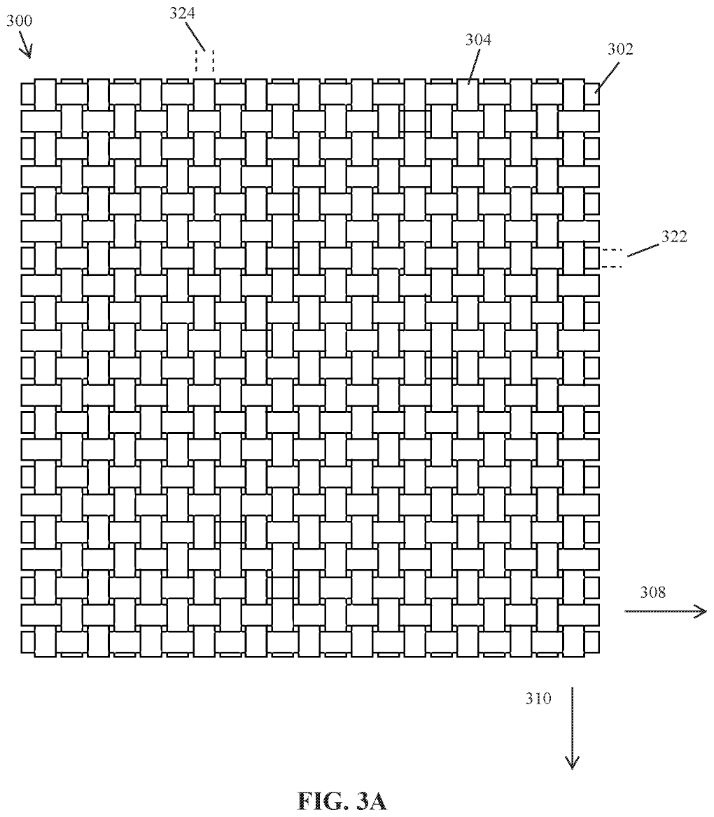

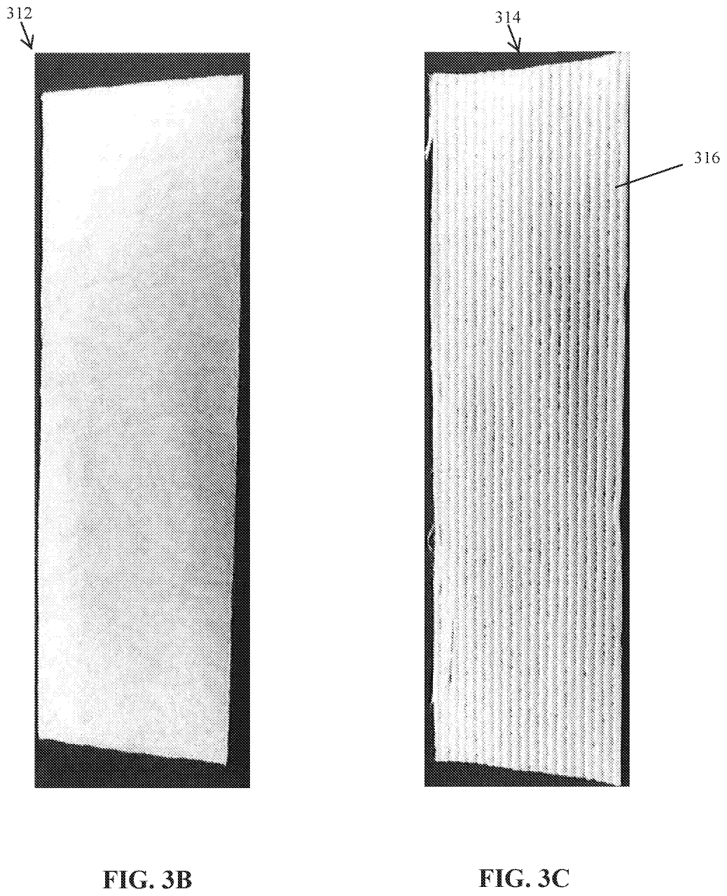

Referring now to FIGS. 3A-3C, FIG. 3A depicts a first embodiment 300 of a composite web. As shown, composite web 300 is a weave that comprises a weft having a plurality of first strips 302 and warp having a plurality of second strips 304. Any nonwoven substrate, whether a laminate structure or a unitary structure, can be slit or cut into strips that may be parallel, zig-zag, sinusoidal, etc. having “n” width which can then be woven into a composite web using known weaving patterns—basket weave, twill, herringbone, etc.—that result in a woven composite web having unique flexural and or fluid management characteristics, different from the parent substrate. Depending on the initial substrates used and the weaving pattern chosen, the resulting composite web can comprise a top sheet, back sheet, surge layer, dryness layer, acquisition/distribution layer, absorbent core, storage core, or a combination thereof.

In the embodiment shown, the first and second strips are arranged such that each of the first strips extends in a first direction 308 and each of the second strips extends in a second direction 310 that is perpendicular to first direction 308. In some embodiments, second strips 304 are disposed at a non-parallel angle relative to first strips 302, such as, for example, an angle that is greater than any one of, or between any two of: 80°, 70°, 60°, 50°, 40°, 30°, 20°, and/or 10°. As shown, first strips 302 and second strips 304 are woven in a basket weave pattern. In other embodiments, first strips 302 and second strips 304 are not woven together, but instead are oriented at a non-parallel angle with respect to each other and are adhered or otherwise bonded to each other. In some embodiments, first strips 302 can be adhered to or otherwise bonded to second strips 304, using, for example, point bonds, adhesive, ultrasonic bonds, pressure bonds, and/or thermal bonds. In other embodiments, first strips 302 need not be bonded to second strips 304, for example, the weave maintains the relative positions of first strips 302 and second strips 304 by utilizing a continuous weft ribbon or strip, which locks up the warp ribbons or strips at the edges of composite web 300 as the weft ribbon or strip is looped back around for the next weft pass through the warp. In some instances, first strips 302 can comprise the continuous weft ribbon or strip and second strips 304 can comprise the warp ribbons or strips. Alternatively, in some embodiments, a light-weight top sheet can be adhered to the surface of composite web 300 to hold composite web 300 together. In other embodiments, ends of strips 302, 304 can be heat sealed, ultrasonically bonded, or otherwise adhered to other strips to keep the ends from separating from composite web 300.

As shown, first strips 302 comprise a material that is different than a material of second strips 304. For example, the depicted first strips 302 each comprise a high loft laminate 312, depicted in FIG. 3B, comprising tissue and superabsorbent polymer (SAP) particles. The depicted second strips 304 each comprise an embossed laminate 314, depicted in FIG. 3C, also comprising tissue and SAP particles, but having a different structure than high loft laminate 312. In other instances, first strips 302 and/or second strips 304 can comprise, for example, a nonwoven web with or without SAP particles, tissue, paper, and/or a through-air bonded polymer nonwoven. By way of example, high loft laminate 312 and embossed laminate 314 each comprise a different multi-layered core construction having a mixture of SAP particles and adhesive disposed between two substrates, for example, layers of tissue. By way of another example, paper for use with first strips 302 and/or second strips 304 can comprise extensible paper, for example paper formed with the Papermorphosis® system from Gruppo X, that can be elongated in the machine direction by up to 4%, 8%, 12%, 16%, or 20% of its relaxed dimension, and/or in the cross direction by up to 4%, 8%, 12%, 16%, or 20% of its relaxed dimension. In some embodiments, first strips 302 can comprise embossed laminate 314 and second strips 304 can comprise high loft laminate 312 for a different outcome of flexibility or fluid management.

In the depicted embodiment, high loft laminate 312 and embossed laminate 314 have different properties, for example stiffness, softness, and absorption, by virtue of having different structures. By way of example, high loft laminate 312 can have a high loft structure with lower density and higher absorption characteristics than embossed laminate 314, allowing high loft laminate 312 to swell more than embossed laminate 314 when absorbing liquid such that, when composite web 300 absorbs liquid, first strips 302 are thicker than second strips 304. Additionally, high loft laminate 312 can be softer and less stiff than embossed laminate 314. Embossed laminate 314 has a linear embossing pattern that defines a plurality of channels 316. Accordingly, when embossed laminate 314 absorbs fluid, the fluid tends to follow channels 316, giving embossed laminate 314 higher fluid movement characteristics than high loft laminate 312.

“Superabsorbent” or “superabsorbent material” or “SAP” refers to a water-swellable, water-insoluble organic or inorganic material capable, under the most favorable conditions, of absorbing at least about 15 times its weight in an aqueous solution containing 0.9 weight percent sodium chloride and, more desirably, at least about 30 times its weight in an aqueous solution containing 0.9 weight percent sodium chloride and, even more desirably, at least about 50 times its weight in an aqueous solution containing 0.9 weight percent sodium chloride. Exemplary superabsorbent polymer material can comprise any superabsorbent polymer particles known from superabsorbent literature, for example such as described in Modern Superabsorbent Polymer Technology, F. L. Buchholz, A. T. Graham, Wiley 1998. For example, the SAP particles may be spherical, spherical-like or irregularly shaped particles, such as sausage shaped particles, or ellipsoid shaped particles of the kind typically obtained from inverse phase suspension polymerizations. The SAP particles can also be optionally agglomerated at least to some extent to form larger irregular particles. In some embodiments, the SAP particles can also have a surface modification, such as a partial or full surface coating, for example to increase the hydrophilicity of the SAP particles.

The SAP materials can be natural, synthetic and modified natural polymers and materials. In addition, the SAP materials can be or include organic compounds such as cross linked polymers. “Cross-linked” is a commonly understood term and refers to any approach for effectively rendering normally water-soluble materials substantially water insoluble, but swellable. Such polymers can include, for example, carboxymethylcellulose, alkali metal salts of polyacrylic acids, polyacrylamides, polyvinyl ethers, hydroxypropyl cellulose, polyvinyl morpholinone, polymers and copolymers of vinyl sulfonic acid, polyacrylates, polyacrylamides, polyvinyl pyridine and the like. Other suitable polymers include hydrolyzed acrylonitrile grafted starch, acrylic acid grafted starch, and isobutylene maleic anhydride copolymers, and mixtures thereof. Organic high-absorbency materials can include natural materials, such as agar, pectin, guar gum and peat moss. In addition to organic materials, superabsorbent materials may also include inorganic materials, such as absorbent clays and silica gels. Suitable examples of SAP include T9030, T9600, T9900, and Saviva polymers from BASF Corporation in Charlotte, N.C.; and W211, W112A, W125, S125D, QX-W1482, QX-W1486, QX-W1504, and QX-W1505 from Nippon Shokubai Co. Ltd, N.A.I.I. in Houston, Tex.; and AQUA KEEP SA50 II, SA55SX II, SA60N II, SA65s, HP500, HP600, and HP 700E from Sumitomo Seika Chemicals Co., Ltd. in Osaka, Japan.

Each of first strips 302 has width 322 and each of second strips 304 has width 324. Widths 322, 324 can be, for example, 5 mm, 10 mm, 15 mm, 20 mm, 25 mm, 30 mm, 35 mm, 40 mm, 45 mm, or 50 mm. As shown, width 322 and width 324 are the same or substantially the same. In some embodiments, width 322 can be different from width 324, such as, for example, a width that is greater than any one of, or between any two of, 10%, 20%, 30%, 40%, 50%, 60%, 70%, 80%, 90%, or 100% of width 324. For example, when first strips 302 comprise high loft laminate 312 and second strips 304 comprise embossed laminate 314, first strips 302 can have a width 322 larger than width 324 to increase the fluid absorption characteristics of composite web 300. By contrast, using second strips 304 that have a width 324 larger than width 322 can increase the fluid movement characteristics of composite web 300. In other examples, such as where first and second strips 302, 304 both comprise a nonwoven web stretchable in one direction, increasing width 322 of first strips 302 can increase the stretchability of composite web 300 in first direction 308, and increasing width 324 of second strips 302 can increase the stretchability of composite web 300 in second direction 310.

In some instances, first and second strips 302, 304 have different thicknesses. First strips can be, in some instances, 5%, 10%, 15%, 20%, 25%, 30%, 35%, 40%, 45%, or 50% thicker than second strips. For example, when first strips 302 comprise high loft laminate 312, having thicker first strips 302 can increase the absorption characteristics of composite web 300 and allow greater swelling of first strips 302 relative to second strips 304. In other embodiments, the thickness of first and second strips 302, 304 can be the same or substantially the same.

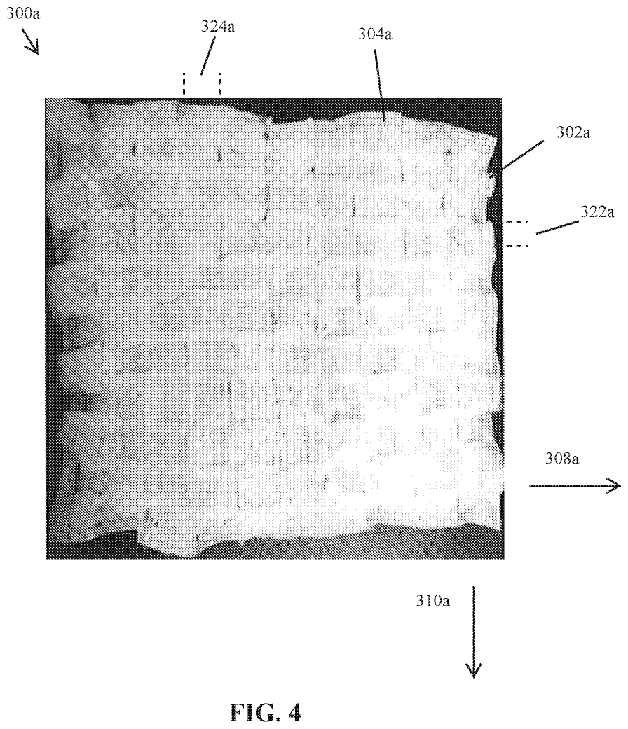

Referring now to FIG. 4, in other embodiments of the present composite webs, the first and second strips can be made of the same material. For example, first and second strips 302a, 304a depicted in FIG. 4 comprise a nonwoven web that is stretchable in one direction but not another direction. Thus, first strips 302a are oriented such that they are stretchable along first direction 308a and second strips 304a are oriented such that they are stretchable along second direction 310a. As used in this disclosure, the term “stretch” and “stretchable” are used interchangeably to define a material or composite that can be elongated by at least 10% of its relaxed dimension, i.e., elongated to at least 1.10 times its relaxed dimension—an elongation of 10%—and that will recover upon release of the applied force at least 10% of its elongation. According to this definition, upon release of the applied force at 10% elongation, the material or composite must recover to at least about a 5% or less elongation. For example, a material or composite is deemed to be “stretchable” if a sample length of 100 centimeters can be elongated to a length of at least 110 centimeters, and upon release of the applied force recovers to a length of not more than about 105 centimeters. Many elastic or stretchable materials or composites can be elongated by more than 10% of their relaxed length, and many of these will recover to, or close to, their original relaxed length upon release of the applied force. These materials can include not only webs of elastic or stretchable films, such as cast or blown films, but also nonwoven fibrous elastic webs such as meltblown elastomeric fibrous nonwoven webs and elastic strands. “Elastic,” “elasticized” and “elasticity” mean that property of a material by virtue of which it tends to recover its original size and shape after removal of a force causing a deformation. Different weave patterns can exhibit different stretch forces depending on how the strips are arranged in the pattern.

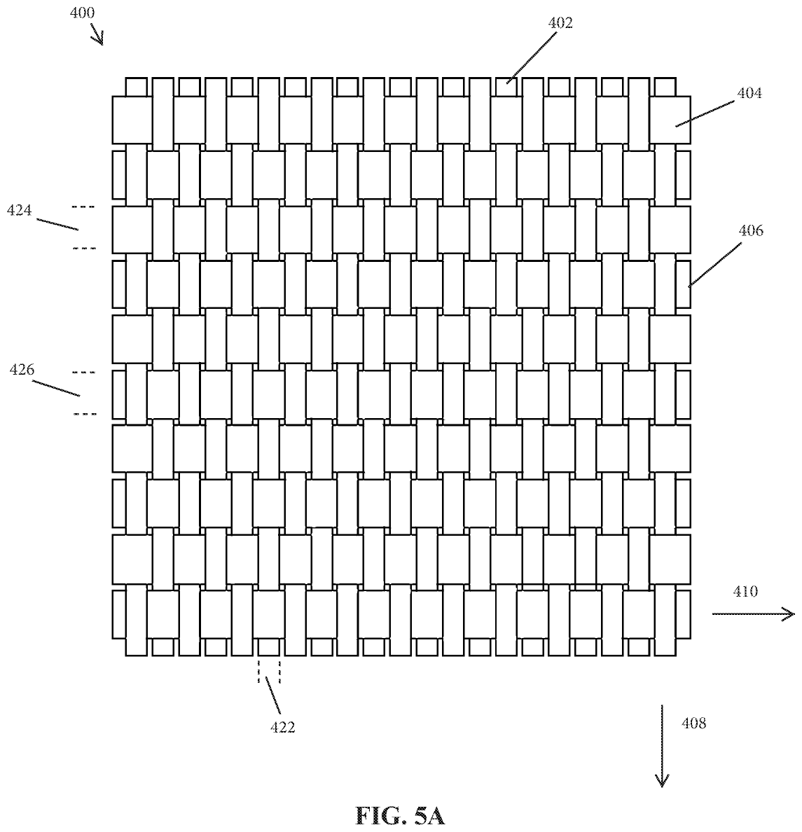

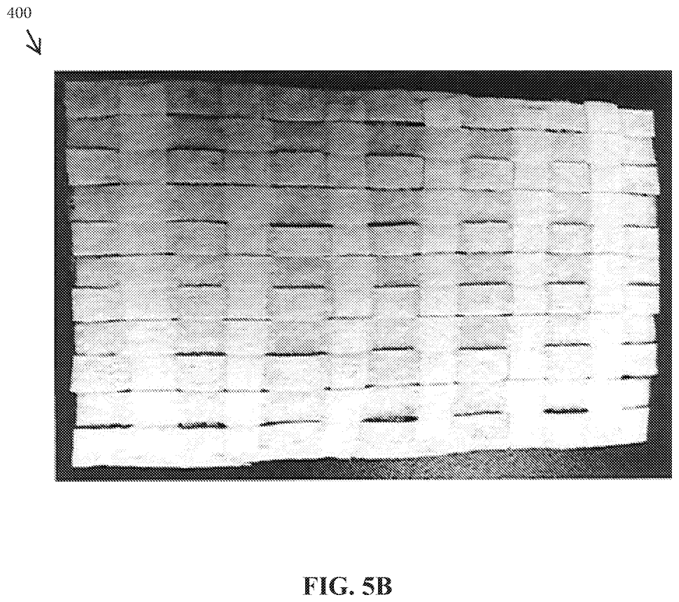

Referring now to FIGS. 5A-5B, FIG. 5A and FIG. 5B depict a second embodiment 400 of a composite web. As shown, composite web 400 is a weave that comprises a warp having a plurality of first strips 402 and a weft having plurality of second strips 404 and a plurality of third strips 406. In the embodiment shown, each of first strips 402 extends in a first direction 408, and each of second and third strips 404, 406 extends in a second direction 410 that is perpendicular to first direction 408. In some embodiments, second and third strips 404, 406 are both disposed at a non-parallel angle relative to the first strips 402, such as, for example, an angle that is greater than any one of, or between any two of: 80°, 70°, 60°, 50°, 40°, 30°, 20°, and/or 10. In other embodiments, second strips 404 are disposed at a first non-parallel angle relative to first strips 402, such as, for example, an angle that is greater than any one of, or between any two of: 80°, 70°, 60°, 50°, 40°, 30°, 20°, and/or 10°, and third strips 406 are disposed at a second non-parallel angle relative to first strips 402, such as, for example, an angle that is greater than any one of, or between any two of: 80°, 70°, 60°, 50°, 40°, 30°, 20°, and/or 10°.

As shown, first, second, and third strips 402, 404, 406 are woven together in a basket weave pattern with a weft comprising adjacent strips that alternate between one of the second strips 404 and one the third strips 406, i.e. each of second strips 404 is adjacent to at least one of the third strips 406 and each of third strips 406 is adjacent to at least one of the second strips 404. In other embodiments, first, second, and third strips 402, 404, 406 are not woven together. In some embodiments, first strips 402 may be adhered to or otherwise bonded to second and third strips 404, 406 using, for example, point bonds, adhesive, ultrasonic bonds, pressure bonds, and/or thermal bonds. In other embodiments, first strips 402 need not be bonded to second and third strips 404, 406, for example, the weave maintains the relative positions of first, second, and third strips 402, 404, 406.

The depicted first and third strips 402, 406 are made of the same first material, and the depicted second strips 404 are made of a different material. For example, each of first and third strips 402, 406 can comprise a laminate that comprises tissue and SAP particles, such as NovaZorb®, an airlaid laminate that incorporates specialty fluff pulp, SAP, and tissue. The laminate, for example, can comprise a multi-layered core construction having a mixture of SAP particles and adhesive disposed between two layers of tissue. Each of second strips 404 can comprise a material used in a conventional ADL, such as a through-air bonded polymer nonwoven, which, in some instances, can have a basis weight of 30, 40, 50, 60, or 70 grams per square meter (gsm). First and third strips 402, 406 accordingly can have higher liquid absorption characteristics than second strips 404, and second strips 404 can have higher liquid permeability and hydrophilic characteristics than first and third strips 402, 406. First strips 402 and/or second strips 406 can comprise, in other instances, for example, a nonwoven web with or without SAP particles, tissue, paper, and/or a through-air bonded polymer nonwoven. In some embodiments, second strips 404 may comprise, for example, a nonwoven web with or without SAP particles, tissue, paper, and/or a laminate comprising tissue and SAP particles.

In other embodiments, composite web 400 comprises a weave having a weft of adjacent strips that has a different arrangement of second and third strips 404, 406, for example such that at least 1, 5, 10, 15, 20, 25, 30, 35, 40, 45, or 50 of second strips 404 are adjacent to at least two other second strips 404, and/or at least 1, 5, 10, 15, 20, 25, 30, 35, 40, 45, or 50 of the third strips 406 are adjacent to at least two other third strips 406. Using a weft having a different arrangement of second and third strips 404, 406 can change the characteristics of composite web. For example, a weft having two of the second strips 404 for every one of the third strips 406 can have increased fluid movement characteristics and decreased fluid absorption characteristics suitable, for example, for use when composite web 400 will have to absorb more liquid bursts, but less liquid overall. In another example, a weft having one of the second strips 404 for every two of the third strips 406 can have decreased fluid movement characteristics and increased fluid absorption characteristics suitable, for example, for use when composite web 400 will have to absorb more liquid overall.

FIG. 5A depicts each of first, second, and third strips 402, 404, 406 having widths 422, 424, and 426 respectively. Widths 422, 424, and 426 can be at least, for example, 5 mm, 10 mm, 15 mm, 20 mm, 25 mm, 30 mm, 35 mm, 40 mm, 45 mm, or 50 mm. As shown, width 422 can be different from widths 424, 426, and widths 424, 426 can be the same or substantially the same. By way of example, widths 424, 426 can be a width that is greater than any one of, or between any two of, 10%, 20%, 30%, 40%, 50%, 60%, 70%, 80%, 90%, or 100% of width 422. In some embodiments, widths 422, 424, and 426 are the same. In other embodiments, widths 422, 424, 426 are each different. For example, width 424 can be larger than width 422 such that second strips 404 will distribute absorbed liquid more rapidly throughout composite web 400 and to first and third strips 402, 406. In other examples, width 422 can be larger than width 424 such that composite web 400 can absorb more liquid.

In some instances, first, second, and third strips 402, 404, 406 are of substantially the same thickness when composite web 400 has not absorbed liquid. In other embodiments, first strips 402, second strips 404, and/or third strips 406 may be of different thicknesses. For example, first and third strips 402, 406 can be 5%, 10%, 15%, 20%, 25%, 30%, 35%, 40%, 45%, or 50% thicker than second strips 404 such that composite web 400 has increased liquid absorption characteristics.

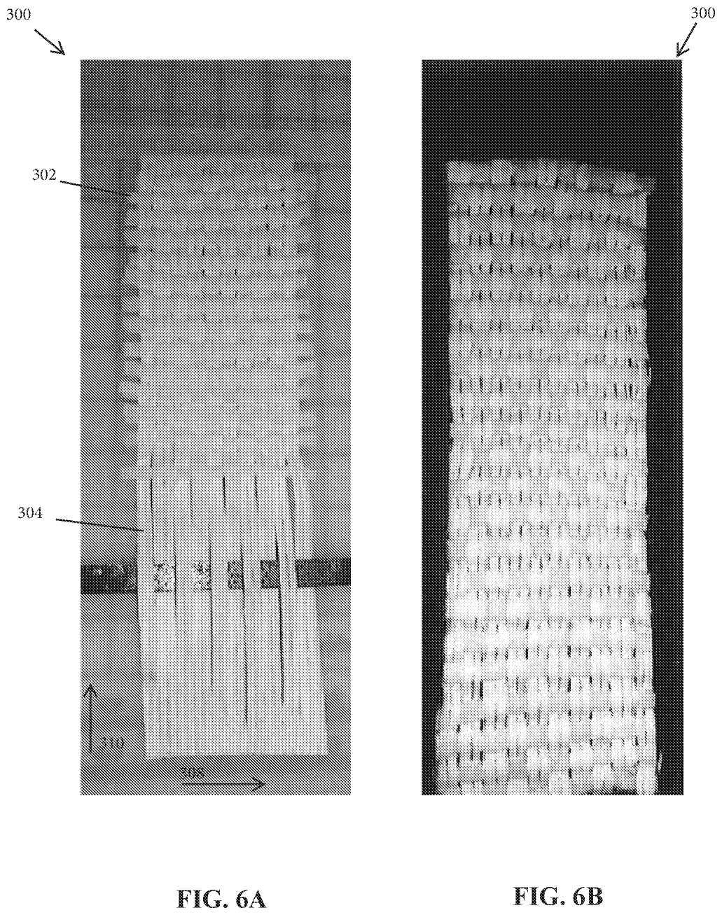

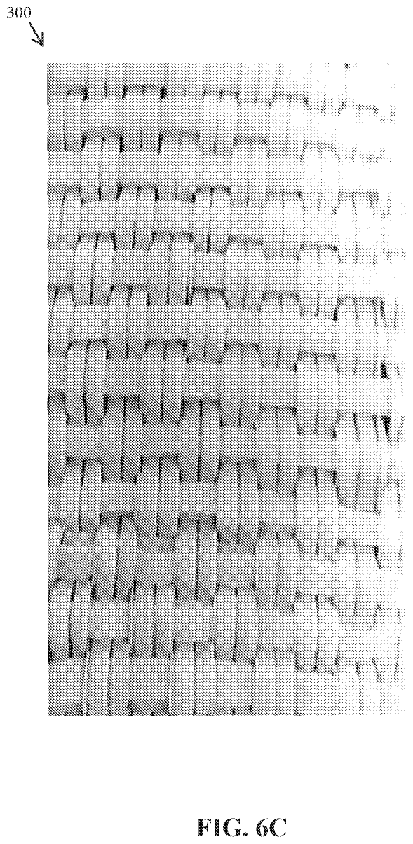

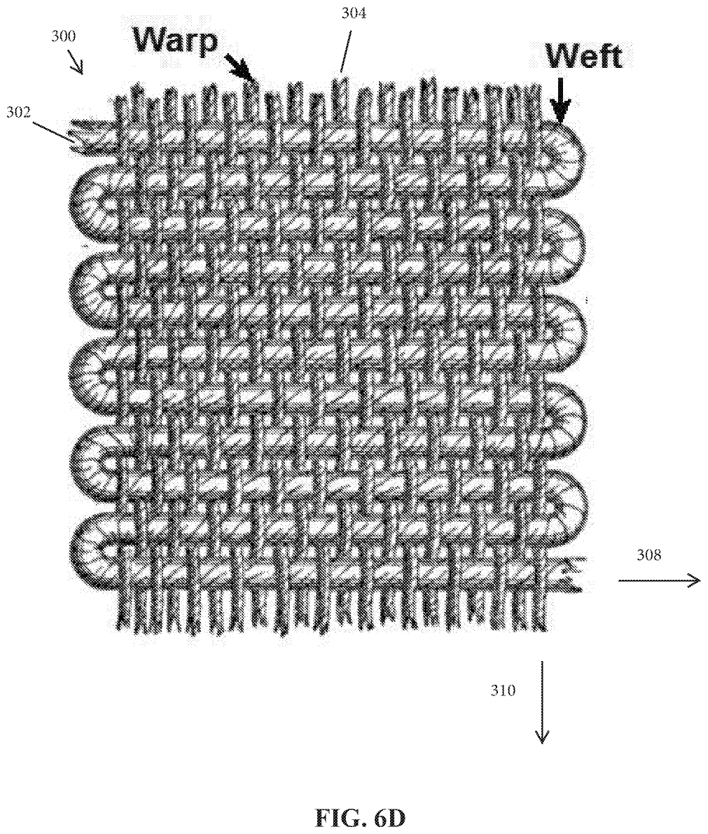

Referring now to FIGS. 6A-6D, FIG. 6A depicts a first technique for producing the present composite webs and FIG. 6D depicts a second technique for producing the present composite webs. In some methods of making the present composite webs, a plurality of first strips are cut from a first material and a plurality of second strips are cut from a second material. The first and second materials can be a suitable material such as, for example, a nonwoven web with or without SAP particles, tissue, a laminate comprising tissue and SAP particles, paper, and/or a through-air bonded polymer nonwoven. The first strips are oriented along a first direction, and the second strips are woven along a second direction non-parallel to the second direction between the first strips, thereby forming a weave. In some embodiments, the second strips are woven in a direction perpendicular to the first strips. In yet further embodiments, the first strips can be adhered to or otherwise bonded using, for example, point bonds, adhesive, ultrasonic bonds, pressure bonds, and/or thermal bonds. In other embodiments, the first strips are not bonded to the second strips, i.e. the weave maintains the relative positions of the first and the second strips. Additionally, in some instances, a plurality of third strips are woven along the second direction between the first strips.

In the technique depicted in FIG. 6A, first strips 302 are cut from high loft laminate 312 of FIG. 3B and second strips 304 are cut from embossed laminate 314 of FIG. 3C. Second strips 304 are oriented along second direction 310, and first strips 302 are woven along first direction 308 between adjacent second strips 304 to create composite web 300 depicted in FIG. 6B and FIG. 6C. In other embodiments, first strips 302 may first be oriented along first direction 308 and second strips 304 may be woven along second direction 310 between adjacent first strips 302. In yet further embodiments, first strips 302 and second strips 304 are not woven together, but instead are oriented at a non-parallel angle with respect to each other and adhered to or otherwise bonded using, for example, point bonds, adhesive, ultrasonic bonds, pressure bonds, and/or thermal bonds.

In the technique depicted in FIG. 6D, second strips 304 are oriented along second direction 310 and comprise the warp of composite web 300. First strips 302 may comprise one or more continuous weft strips. The continuous weft strip(s) is woven along first direction 308 between adjacent second strips 304 in a series of passes. At the end of a pass, the continuous weft strip is looped back around for the next weft pass. In this manner, first strips 302 lock up second strips 304 at the edges of composite web 300 so as to maintain the relative positions of strips 302, 304. In some embodiments, first and second strips 302, 304 can be adhered to or otherwise bonded using, for example, point bonds, adhesive, ultrasonic bonds, pressure bonds, and/or thermal bonds to further maintain the relative positions of strips 302, 304.

The present composite webs can have more desirable properties than webs comprised solely of their constituent substrates. For example, embodiments comprising a weave can be more flexible than both of the constituent strips comprising the weave's weft and warp. Additionally, the present composite webs can have fluid management characteristics better suited for absorbent articles than the composite webs' constituent substrates. By way of example, some embodiments of the present composite webs comprise strips having high fluid absorption characteristics, such as high loft laminates, and strips having high fluid management characteristics, such as laminates having a plurality of channels. The strips with high fluid movement characteristics can better disperse fluid throughout the composite web and deliver fluid to the strips having high fluid absorption characteristics, thereby combining the high absorption and high fluid movement characteristics of the constituent strips. By way of another example, other embodiments of the present composite webs comprise strips having high fluid absorption characteristics, such as NovaZorb®, and strips having high permeability, such as strips comprising a through-air bonded polymer nonwoven. The high permeability strips can acquire a gush of liquid as rapidly as it is added to the composite web and transfer the liquid to the strips with high absorption characteristics to partially return the high permeability strips to their initial state for subsequent insults of liquids. Thus, the composite web can combine the high liquid absorption characteristics and high permeability of its constituent strips to allow for more rapid liquid absorption and to avoid leakage. In another embodiment for an absorbent core the composite web can be folded back onto itself such as a c-fold, z-fold, g-fold, etc. to multiply the fluid management ability of the composite web. Additionally, yet further embodiments of the present composite webs are stretchable in multiple directions by virtue of the composite web's weave structure, notwithstanding the constituent strips being stretchable in only one direction.

The present composite webs can be used in various absorbent articles, for example, as part of underwear 10, or, in other examples, as part of brief 100. In some instances, the article does not have absorbent core 200, and chassis 104 of brief 100 comprises composite web 300 depicted in FIG. 3A. In another such example of an absorbent article, the article does not include ADL 204 or absorbent core 200, and the chassis 104 comprises composite web 400 depicted in FIG. 4A. In some instances, the article comprises a composite web 300, 400 disposed between topsheet 196 and backsheet 192. In other instances, the article can comprise a composite web 300, 400 and omit topsheet 196 and/or backsheet 192.

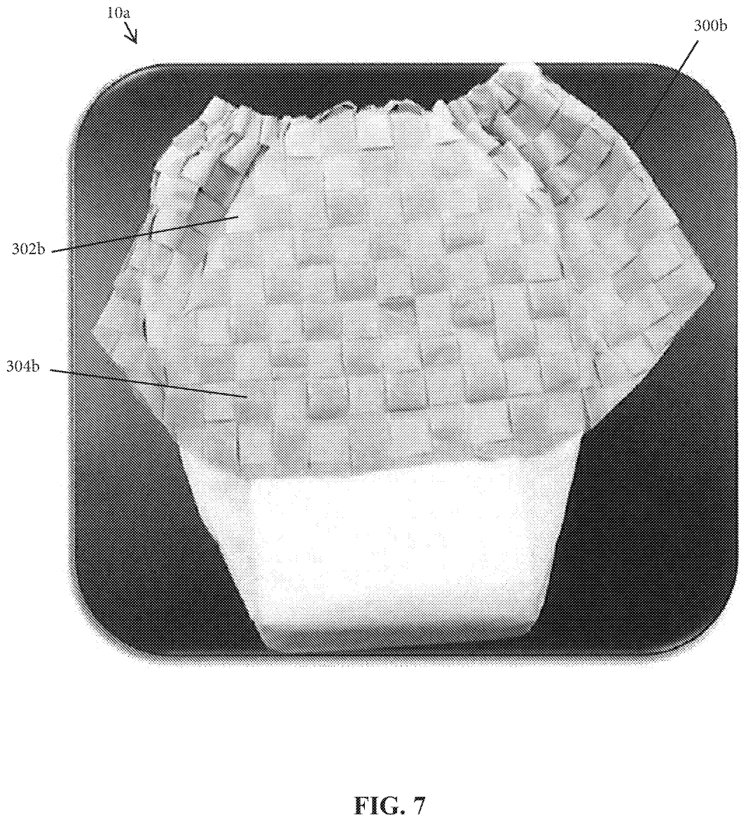

FIG. 7 depicts a perspective view of an embodiment of the present absorbent articles, specifically adult protective underwear 10a, in a closed configuration. Underwear 10a is substantially similar in many respects to underwear 10 and, therefore, the differences will primarily be described here. For brevity and clarity, many of the reference numerals for features that are similar in underwear 10 to those in underwear 10a are therefore omitted in FIG. 6, but such features should be understood to be similar to those in underwear 10 for at least the depicted embodiment of underwear 10a, unless otherwise described for underwear 10a.

The primary difference in underwear 10a relative to underwear 10 is that front and rear waist portions 18, 22 of chassis 14 of underwear 10a incorporate composite web 300a. Composite web 300b is substantially similar in many respects to composite web 300. For example, the depicted composite web 300b comprises a weave having a weft that comprises a plurality of first strips 302b and warp that comprises a plurality of second strips 304b oriented perpendicular to first strips 302b. In the embodiment shown, first strips 302b and second strips 304b are made of laminated paper.

In other embodiments, chassis 14 of underwear 10a may comprise composite web 300 depicted in FIG. 3A and described above, disposed between topsheet 34 and backsheet 30. As described above, first strips 302 swell more than second strips 304 when the depicted composite web 300 absorbs liquid. Thus, first strips 302 provide higher points of contact with topsheet 34 when composite web 300 absorbs liquid, thereby lifting topsheet 34 away from second strips 304 and maintaining a drier topsheet 34. In some embodiments, the chassis 14 of underwear 10a may comprise composite web 400 depicted in FIG. 5A and described above, disposed between topsheet 34 and backsheet 30. In some instances, underwear 10a can omit the topsheet 34 and/or backsheet 30.

The present backsheets are typically liquid-impermeable and can include, for example, an inner liquid-impermeable film and an outer nonwoven backsheet that can be a nonwoven fabric. A “film” is a membrane-like layer of material formed of one or more polymers, which does not have a form consisting predominately of a web-like structure of fibers and/or other fibers. In some embodiments of the present articles, backsheet or outer cover 30, 192 can be breathable, for example, an inner liquid-impermeable film of backsheet 30, 192 can comprise a breathable film. The terms “breathable,” “breathable film,” “breathable laminate” or “breathable outer cover material” or “breathable backsheet” refers to a film, laminate, or outer cover material having a water vapor transmission rate (“WVTR”) of at least about 300 grams/m²/24 hours. Breathable materials typically rely on molecular diffusion of vapor, and are substantially liquid impermeable. “Nonwoven” fabrics, according to an INDA definition, are broadly defined as sheet or web structures bonded together by entangling fiber or filaments, and by perforating films, mechanically, thermally, or chemically. They are flat, porous sheets that are made directly from separate fibers or from molten plastic or plastic film. They are not made by weaving or knitting and do not require converting the fibers to yarn. The basis weight of nonwoven fabrics is usually expressed as gsm or grams per square meter. “Nonwoven backsheet” is a backing substrate layer in the outer cover; a nonwoven backsheet is most often a nonwoven layer facing away from the wearer.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

By way of example, FIG. 3A depicts composite web 300 as comprising first and second strips 302, 304 and FIG. 5A depicts composite web 400 as comprising first, second, and third strips 402, 404, 406. However, in other embodiments, composite web 300 and/or composite web 400 can comprise additional strips, for example, a plurality of fourth strips, fifth strips, sixth strips, and/or seventh strips, each plurality of strips comprising a nonwoven web with or without SAP particles, tissue, a laminate comprising tissue and SAP particles, paper, and/or a through-air bonded polymer nonwoven. By way of another example, FIG. 7 depicts underwear 10a as comprising composite web 300b incorporated into front and rear waist portions 18, 22 of chassis 14. However, in other embodiments, the present absorbent articles can include chassis 14 that comprises front waist portion 18, rear waist portion 22, and crotch portion 26, all comprising a composite web 300b. Additionally, while the present absorbent articles are described with reference to adult incontinence briefs and protective underwear, the present absorbent articles can also comprise baby diapers and training pants. For example, baby diapers and training pants can also be provided with one or more of the present composite webs. Additionally, any of the present absorbent articles can comprise or omit any of the various other elements and variations described in connection with the various chassis configurations, such as, for example, absorbent core(s), ADL(s), backsheet(s), topsheet(s), closure members, and/or the like. For example, chassis 14 of any of the present absorbent articles can comprise any of the various composite webs described above, such as composite web 300a depicted in FIG. 4.

The claims are not intended to include, and should not be interpreted to include, means-plus- or step-plus-function limitations, unless such a limitation is explicitly recited in a given claim using the phrase(s) “means for” or “step for,” respectively.

1. A composite web comprising:
a plurality of first strips of tissue, paper, or nonwoven web extending in a first direction; and
a plurality of second strips coupled to the first strips and extending in a second direction disposed at a non-parallel angle relative to the first direction.

2. The composite web of claim 1, wherein the first strips and the second strips are woven together.

3. The composite web of claim 1, wherein the first strips comprise a laminate that includes tissue and superabsorbent polymer (SAP) particles.

4. The composite web of claim 1, wherein the second strips comprise a laminate web that includes tissue and superabsorbent polymer (SAP) particles.

5. The composite web of claim 1, wherein each of the second strips comprises one or more channels extending along the second direction.

6. The composite web of claim 1, wherein the second plurality of strips comprise a through-air bonded polymer nonwoven web.

7. The composite web of claim 1, further comprising a plurality of third strips extending in a third direction disposed at a non-parallel angle relative to the first direction.

8. The composite web of claim 7, wherein:
the first strips and the third strips comprise a laminate comprising tissue and SAP particles; and
the third direction is the same as the second direction.

9. The composite web of claim 7, the first, second, and third strips woven together such that the composite web comprises a weave having:
a warp comprising the first strips; and
a weft comprising the second and third strips, each of the second strips adjacent to at least one of the third strips.

10. The composite web of claim 1, wherein:
the first strips are configured to stretch more in the first direction than in a direction perpendicular to the first direction; and
the second strips are configured to stretch more in the second direction than in a direction perpendicular to the second direction.

11. The composite web of claim 1, wherein the first strips are at least 5% thicker than the second strips.

12. The composite web of claim 1, wherein the first plurality of strips are bonded to the second plurality of strips.

13. An absorbent article comprising:
a chassis having opposing front and rear waist portions, a crotch portion extending longitudinally between the front and rear waist portions, where a first end of the front waist portion is configured to be coupled to a first end of the rear waist portion and a second end of the front waist portion is configured to be coupled to a second end of the rear waist portion to define a closed configuration in which the front and rear waist portions cooperate to encircle and define a waist opening, a left side of the chassis defines a first leg opening, and a right side of the chassis defines a second leg opening;
wherein the chassis comprises at least one composite web of claim 1.

14. The absorbent article of claim 13, wherein:
the chassis further comprises a topsheet disposed on the at least one composite web;
the first strips swell to a first thickness when the first strips absorb moisture; and
the first strips and second strips are woven together such that the topsheet contacts the first strips and not the second strips when the first strips have the first thickness.

15. A method of making a composite web, the method comprising:
orienting a plurality of first strips of tissue or nonwoven web in a first direction; and
weaving a plurality of second strips of tissue or nonwoven web, in a second direction disposed at a non-parallel angle relative to the first direction, between the first strips.
